SEQUENCE LISTING (44 sequences)

SEQ ID NO: 1 - DNA, 22 bp, Artificial Sequence
  FWLMPCR Primer
acagtagggctgtgcttactgc

SEQ ID NO: 2 - DNA, 22 bp, Artificial Sequence
  REVLMPCR Primer
ctcattttcagcctcaatcctc

SEQ ID NO: 3 - DNA, 22 bp, Artificial Sequence
  FW2rs16994908 Primer
cttcaccatttttgcatgtacc

SEQ ID NO: 4 - DNA, 22 bp, Artificial Sequence
  REVrs16994908 Primer
ttgcaaccacatttgaagtgac

SEQ ID NO: 5 - DNA, 22 bp, Artificial Sequence
  FWrs12688573 Primer
aaagtcgcacagataccctctc

SEQ ID NO: 6 - DNA, 22 bp, Artificial Sequence
  REVrs12688573 Primer
cttttctgtcttgccattagcc

SEQ ID NO: 7 - DNA, 22 bp, Artificial Sequence
  FWrs11117557_3 Primer
actgcatctgcagagaaacaac

SEQ ID NO: 8 - DNA, 23 bp, Artificial Sequence
  REVrs11117557_3 Primer
aacagttgtgaaactacgtcagg

SEQ ID NO: 9 - DNA, 22 bp, Artificial Sequence
  FWrs7052829 Primer
ttatgggaagaatccactccag

SEQ ID NO: 10 - DNA, 22 bp, Artificial Sequence
  REVrs7052829_2 Primer
agtagcagcaacagcaacaaag

SEQ ID NO: 11 - DNA, 20 bp, Artificial Sequence
  FWrs7052654_rpt Primer
cagggcagggatgattagag

SEQ ID NO: 12 - DNA, 22 bp, Artificial Sequence
  REVrs7052654_rpt Primer
agaaaggaagagatgcatggac

SEQ ID NO: 13 - DNA, 22 bp, Artificial Sequence
  FWrs6626955_6 Primer
tcccttgtgttcatggagtatg

SEQ ID NO: 14 - DNA, 22 bp, Artificial Sequence
  REVrs6626955_6 Primer
aacaggagcttcttcctgattg

SEQ ID NO: 15 - DNA, 22 bp, Artificial Sequence
  FWrs2761622_2 Primer
aaatgaaatgcaccttccagag

SEQ ID NO: 16 - DNA, 22 bp, Artificial Sequence
  REVrs2761622_2 Primer
gcacttgtttcacaggtacagc

SEQ ID NO: 17 - DNA, 22 bp, Artificial Sequence
  FWrs1805422 Primer
gtagcagtagtgcgtttgttgg

SEQ ID NO: 18 - DNA, 22 bp, Artificial Sequence
  REVrs1805422 Primer
tttcctatagccaaacgtgtcc

SEQ ID NO: 19 - DNA, 24 bp, Artificial Sequence
  FWrs1265401 Primer
gggtatgggtttaacataggacag

SEQ ID NO: 20 - DNA, 21 bp, Artificial Sequence
  REVrs1265401 Primer
gacttacgggctgcttctcac

SEQ ID NO: 21 - DNA, 22 bp, Artificial Sequence
  FWrs1265397 Primer
gcatgcgtgtcttactccatag

SEQ ID NO: 22 - DNA, 22 bp, Artificial Sequence
  REVrs1265397 Primer
aagctctgtcagtgtgatgtgg

SEQ ID NO: 23 - DNA, 25 bp, Artificial Sequence
  FWDrs25699 Primer
gccagaggctatttccctaacttac

SEQ ID NO: 24 - DNA, 24 bp, Artificial Sequence
  REVrs25699 Primer
tgatgacgaactctggaatttgac

SEQ ID NO: 25 - DNA, 23 bp, Artificial Sequence
  FWDrs4949 Primer
agagtgcttttgttgggatgtac

SEQ ID NO: 26 - DNA, 22 bp, Artificial Sequence
  REV_2rs4949 Primer
attacacacataggtggcacta

SEQ ID NO: 27 - DNA, 22 bp, Artificial Sequence
  FWDrs1442280 Primer
agacattgcaaacatccagaac

SEQ ID NO: 28 - DNA, 22 bp, Artificial Sequence
  REVrs1442280 Primer
atgcagtcagccaggtaataga

SEQ ID NO: 29 - DNA, 24 bp, Artificial Sequence
  FWDrs16994869 Primer
tgaacagtcacttgacatccaaag

SEQ ID NO: 30 - DNA, 24 bp, Artificial Sequence
  REVrs16994869 Primer
gattggaggaggcagagaaatagt

SEQ ID NO: 31 - DNA, 22 bp, Artificial Sequence
  FWrs29284_int9 Primer
ctctggtacctgaccaaaggag

SEQ ID NO: 32 - DNA, 22 bp, Artificial Sequence
  REVrs29284_int9 Primer
aaagcagtaagcacagccctac

SEQ ID NO: 33 - DNA, 22 bp, Artificial Sequence
  FWrs29288_int13 Primer
catgccattcattcttatggtg

SEQ ID NO: 34 - DNA, 22 bp, Artificial Sequence
  REVrs29288_int13 Primer
aatcctaactctccaggccttc

SEQ ID NO: 35 - DNA, 22 bp, Artificial Sequence
  FWrs25707_ex5 Primer
cctgccacaaaagatactttcc

SEQ ID NO: 36 - DNA, 21 bp, Artificial Sequence
  REVrs25707_ex5 Primer
ttctccattgctcttgcaaac

SEQ ID NO: 37 - DNA, 22 bp, Artificial Sequence
  FWI304N_ex10 Primer
acagtagggctgtgcttactgc

SEQ ID NO: 38 - DNA, 22 bp, Artificial Sequence
  REVI304N_ex10 Primer
ctcattttcagcctcaatcctc

SEQ ID NO: 39 - DNA, 22 bp, Artificial Sequence
  FW rs29286_int12 Primer
gtggcttcatcagttgtagcag

SEQ ID NO: 40 - DNA, 22 bp, Artificial Sequence
  REVrs29286_int12 Primer
cacatacccacaaacactcctc

SEQ ID NO: 41 - DNA, 23 bp, Artificial Sequence
  FWrs5904816_int14 Primer
gcacatcaaggtttgaacttagg

SEQ ID NO: 42 - DNA, 22 bp, Artificial Sequence
  REVrs5904816_int14 Primer
cagagacgtttcaggggtaatc

SEQ ID NO: 43 - DNA, 22 bp, Artificial Sequence
  FWrs25704_ex17 Primer
ggaaggtcatttccatgtatgc

SEQ ID NO: 44 - DNA, 21 bp, Artificial Sequence
  REVrs25704_ex17 Primer
aaaaccaaaccccaacacttc

(19) United States
(12) Patent Application Publication
(10) Pub. No.: US 2010/0093986 A1
(43) Pub. Date: Apr. 15, 2010

(54) METHODS OF DIRECT GENOMIC SELECTION USING HIGH DENSITY OLIGONUCLEOTIDE MICROARRAYS

(76) Inventors: Michael E. Zwick, Atlanta, GA (US); David T. Okou, Norcross, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/524,252

(22) PCT Filed: Feb. 4, 2008
(86) PCT No.: PCT/US08/52887

§ 371 Date: Sep. 25, 2009

Related U.S. Application Data
(60) Provisional application No. 60/899,159, filed on Feb. 2, 2007.
(60) Provisional application No. 60/979,432, filed on Oct. 12, 2007.

(51) Int. Cl. C07H 1/06

(52) U.S. Cl. 536/23.1

(57) ABSTRACT

The present disclosure encompasses methods (hereinafter termed ‘Microarray-based Genomic Selection’ (MGS), capable of isolating user-defined unique genomic sequences from complex eukaryotic genomes.

### RELATED APPLICATIONS/PATENTS

This application claims priority to provisional U.S. application Ser. No. 60/899,159 filed Feb. 2, 2007 and to provisional U.S. application Ser. No. 60/979,432 filed Oct. 12, 2007, the contents of which are hereby expressly incorporated herein by reference.

### STATEMENT REGARDING FEDERALLY SPONSORED RESEARCH OR DEVELOPMENT

This invention was made with government support under NIH Grant No. RO1 MH076439-01 awarded by the U.S. National Institutes of Health of the United States government. The government has certain rights in the invention

### BACKGROUND

Technological innovation in DNA sequencing offers the promise of a more comprehensive, cost effective, and systematic ascertainment of genetic variation (Cutler et al., Genome Res. 11, 1913-25 (2001); Margulies at al. Nature 437, 376-80 (2005); Shendure et al., Nat. Rev. Genet. 5, 335-44 (2004); Shendure at al., Science 309, 1728-32 (2005); Zwick et al., Genome Biol. 6, R10 (2005)). A major bottleneck, however, lies in isolating the target DNA to be sequenced. Complex eukaryotic genomes, like the human genome, are too large to explore without complexity reduction using methods that directly amplifies specific sequences. Current approaches for target DNA isolation include short PCR (Hinds et al. Science 307, 1072-9 (2005); Sjoblom et al., Science 314, 268-74 (2006)); long PCR (Cutler et al., Genome Res. 11, 1913-25 (2001); Zwick et al., Genome Biol. 6, R10 (2005)), fosmid library construction and selection (Raymond et al., Genomics 86, 759-66 (2005)), TAR cloning (Raymond et al., Genome Res. 12, 190-197 (2002); Kouprina et al., Methods Mol. Biol. 349, 85-101 (2006)), selector technology (Dahl et al., Proc. Natl. Acad. Sci. U.S.A. 104, 9387-92 (2007)), and direct genomic selection with bacterial artificial chromosomes (BACs) (Bashiardes et al., Nat. Methods 2, 63-9 (2005)). PCR using primer pairs complementary to specific genomic regions of interest is still the most common method sample preparation, but it is difficult to scale to large genomic regions, is labor intensive, and when primers are multiplexed, is subject to failure or artifacts. Random clone-based methods offer the advantage of obtaining complete haplotypes, but remain relatively expensive to scale.

Direct genomic selection, using BAC clones as hybridization “hooks”, has previously demonstrated the ability to isolate specific genomic regions without requiring specific amplification (Bashiardes et al., Nat Methods 2, 63-9 (2005)), but its adoption has been limited. Because BAC clones consist of a great deal of highly repetitive sequences, a number of protocol steps are required to minimize the enrichment of these types of sequences. Furthermore, because a single BAC is the unit of selection, isolating discontiguous unique sequence regions from across the genome would require multiple BACs. Finally, the existing protocol depends upon the presence of restriction sites adjacent to the targeted regions of interest that produce sticky ends for the ligation of generic adaptors. This acts to limit coverage in regions lacking these restriction sites. While random shearing followed by repair was mentioned as a possible alternative approach, it was not demonstrated (Bashiardes et al., Nat Methods 2, 63-9 (2005)).

### SUMMARY

The present disclosure encompasses methods (hereinafter termed ‘Microarray-based Genomic Selection’ (MGS)), capable of isolating user-defined unique genomic sequences from complex eukaryotic genomes. The MGS protocol of the disclosure includes, but is not limited to, the following steps: physical shearing of genomic DNA to create random fragments with an average size of 300 bp; end repairing of the fragments that may include, but is not limited to, adding 3′-A overhangs, followed by ligation to unique adaptors with a complementary T nucleotide overhangs; fragment hybridizing and capture using a custom high-density oligonucleotide microarray of complementary sequences identified from a reference genome sequence; elution of fragments bound to the probes, and amplification of selected fragments through one round of PCR using adaptors as a single set of primers/template.

The present disclosure, therefore, provides methods of isolating user-defined unique gene sequences from complex eukaryotic genomes comprising isolating genomic from a human or animal, shearing of the genomic DNA into fragments, repairing the genomic DNA fragments, ligating adapters to the genomic DNA fragments, hybridizing the genomic DNA fragments to oligonucleotides of interest of a high density long oligonucleotide microarray, eluting of the genomic DNA fragments bound to oligonucleotides of interest on the microarray, and amplifying the eluted DNA fragments.

In the various embodiments of the disclosure, the methods therein may further comprise resequencing of the eluted DNA fragments.

In one embodiment of the disclosure, the shearing may be physical shearing. In some embodiments of the disclosure, the shearing can be selected from sonication, nebulization, or a combination thereof.

In the embodiments of the disclosure, the repairing step includes, but is not limited to, using blunt end formation and phosphorylation reactions to repair the genomic DNA fragments.

In the embodiments of the methods of the disclosure, the adaptors may be blunt-end ligated to the genomic DNA fragments and the adapters may not substantially self ligate, are unique relative to the DNA genome, and are complimentary to one another.

In one advantageous embodiment of the disclosure the adaptors may have the nucleotide sequences according to SEQ ID NOs: 1 and 2.

The present disclosure further provides an embodiment of a method of isolating user-defined unique gene sequences from complex eukaryotic genomes comprising, isolating genomic from a human or animal, shearing the genomic DNA into fragments, wherein the shearing is physical shearing selected from sonication, nebulization, or a combination thereof, repairing the genomic DNA fragment, wherein repairing is selected from includes using blunt end formation and adding 3′-A extensions to the genomic DNA fragments, ligating a plurality of adapters to the genomic DNA fragments, and wherein the adapters do not substantially self ligate, are unique relative to the DNA genome, and are complimentary to one another, and wherein the adaptors have the nucleotide sequences according to SEQ ID NOs: 1 and 2, hybridizing the genomic DNA fragments to oligonucleotides of interest of a high density long oligonucleotide microarray, eluting of the genomic DNA fragments bound to oligonucleotides of interest on the microarray; amplifying the eluted DNA fragments and resequencing of the eluted DNA fragments.

### BRIEF DESCRIPTION OF THE FIGURES

FIG. 1 illustrates a schema for a method of microarray-based genomic selection (MGS) and resequencing of complex genomes. In this schema, sheared genomic fragments may be repaired and ligated to generic adaptors. Hybridization to a custom designed high-density oligonucleotide microarray can allow the capture of the target DNA regions. The selected target DNA is eluted and amplified using a one step PCR and a single primer pair/template. Resequencing of the amplified target may be conducted with resequencing arrays analyzed with RATOOLS™.

FIG. 2 illustrates the genomic regions (50 kb, 304 kb) resequenced in two MGS validation experiments. Targeted sequences included both coding and unique non-coding genome sequences.

FIG. 3 illustrates resequencing hybridization results for TR91 (A) and DM316 (B) samples. The large absence of hybridization on the DM316 array is the result of a large deletion of much of the FMR1 locus.

FIG. 4 illustrates the results of quantitative PCR assay measuring the extent of enrichment after a single round of microarray-based genomic selection (MGS). Treatment 1 was a whole genome amplified sample that was passed through the entire MSG protocol, but never hybridized to an array. Treatment 2 was a whole genome amplified sample processed through the entire MGS protocol. The DNA from treatment 2 had a cycle threshold of 15 while the cycle threshold for treatment 1 was 25.

FIG. 5 illustrates amplified DNA from BAC 49K19 after having been hybridized to genomic selection microarray at Nimblegen (Madison, Wis.). PCR amplification was accomplished using generic adapter primers to compare two different methods of genomic DNA fragmentation (nebulization and sonication). N=Nebulized sample; S=Sonicated sample.

FIG. 6 illustrates PCR results for Samples 2, 3, 6 and 7. Eluted refers to samples that were sonicated, end-repaired, adapters ligated, and hybridized to a genomic selection array (Nimblegen, Madison, Wis.). Ligated were control samples (sonication, repair and ligation, but not hybridized to a chip)

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Embodiments of the present disclosure will employ, unless otherwise indicated, techniques of synthetic organic chemistry, biochemistry, biology, molecular biology, and the like, which are within the skill of the art. Such techniques are explained fully in the literature.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein cited references”), as well as each document or reference cited in each of the herein-cited references (including any manufacturer's specifications, instructions, etc.), is hereby expressly incorporated herein by reference.

The methods of this disclosure are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It must be noted that, as used in the specification and the appended claims, the singular forms “a,” “an,” and “the” include plural referents unless the context clearly dictates otherwise. Thus, for example, reference to “a support” includes a plurality of supports.

In this specification and in the claims that follow, reference will be made to a number of terms that shall be defined to have the following meanings unless a contrary intention is apparent. As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DEFINITIONS

In describing and claiming the disclosed subject matter, the following terminology will be used in accordance with the definitions set forth below.

In accordance with the present disclosure there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” (B. D. Hames & S. J. Higgins eds. (1985)); “Transcription and Translation” (B. D. Hames & S. J. Higgins eds. (1984)); “Animal Cell Culture” (R. I. Freshney, ed. (1986)); “Immobilized Cells and Enzymes” (IRL Press, (1986)); B. Perbal, “A Practical Guide To Molecular Cloning” (1984), each of which is incorporated herein by reference.

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Qβ replicase amplification (QβRA); self-sustained replication (3SR); and NASBA (nucleic acid sequence-based amplification), which can be performed on RNA or DNA as the nucleic acid sequence to be amplified.

A “fragment” of a molecule such as a protein or nucleic acid is meant to refer to any portion of the amino acid or nucleotide genetic sequence.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide includes conservatively modified variants. A substituted or inserted amino acid residue may or may not be one encoded by the genetic code. A variant of a polypeptide may be naturally occurring, such as an allelic variant, or it may be a variant that is not known to occur naturally.

Modifications and changes can be made in the structure of the polypeptides of this disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptides as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, at al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, at al., Protein Sci., 2: 395-403, 1993).

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this disclosure, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

As used herein, the term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. Polynucleotide encompasses the terms “nucleic acid,” “nucleic acid sequence,” or “oligonucleotide” as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotides nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alias.

By way of example, a polynucleotide sequence of the present disclosure may be identical to the reference sequence, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

The term “codon” means a specific triplet of mononucleotides in the DNA chain. Codons correspond to specific amino acids (as defined by the transfer RNAs) or to start and stop of translation by the ribosome.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

As used herein, the term “hybridization” refers to the process of association of two nucleic acid strands to form an antiparallel duplex stabilized by means of hydrogen bonding between residues of the opposite nucleic acid strands.

The term “immunologically active” defines the capability of the natural, recombinant or synthetic bioluminescent protein, or any oligopeptide thereof, to induce a specific immune response in appropriate animals or cells and to bind with specific antibodies. As used herein, “antigenic amino acid sequence” means an amino acid sequence that, either alone or in association with a carrier molecule, can elicit an antibody response in a mammal. The term “specific binding,” in the context of antibody binding to an antigen, is a term well understood in the art and refers to binding of an antibody to the antigen to which the antibody was raised, but not other, unrelated antigens.

As used herein the term “isolated” is meant to describe a polynucleotide, a polypeptide, an antibody, or a host cell that is in an environment different from that in which the polynucleotide, the polypeptide, the antibody, or the host cell naturally occurs.

“Optional” or “optionally” means that the subsequently described circumstance may or may not occur, so that the description includes instances where the circumstance occurs and instances where it does not.

The term “array” encompasses the term “microarray” and refers to an ordered array presented for binding to polynucleotides and the like.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this disclosure.

An “array” includes any two-dimensional or substantially two-dimensional (as well as a three-dimensional) arrangement of addressable regions including nucleic acids (e.g., particularly polynucleotides or synthetic mimetics thereof) and the like. Where the arrays are arrays of polynucleotides, the polynucleotides may be adsorbed, physisorbed, chemisorbed, and/or covalently attached to the arrays at any point or points along the nucleic acid chain.

A substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than about 20 cm² or even less than about 10 cm² (e.g., less than about 5 cm², including less than about 1 cm² or less than about 1 mm² (e.g., about 100 μm², or even smaller)). For example, features may have widths (that is, diameter, for a round spot) in the range from about 10 μm to 1.0 cm. Non-round features may have area ranges equivalent to that of circular features with the foregoing width (diameter) ranges.

Arrays can be fabricated using drop deposition from pulse-jets of either polynucleotide precursor units (such as monomers), in the case of in situ fabrication, or the previously obtained nucleic acid. Such methods are described in detail, for example, in U.S. Pat. No. 6,242,266, U.S. Pat. No. 6,232,072, U.S. Pat. No. 6,180,351, U.S. Pat. No. 6,171,797, and U.S. Pat. No. 6,323,043. As already mentioned, these references are incorporated herein by reference.

An array “package” may be the array plus a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and ‘lower” are used in a relative sense only.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular probe sequence. Array features are typically, but need not be, separated by intervening spaces. In the case of an array in the context of the present application, the “probe” will be referenced in certain embodiments as a moiety in a mobile phase (typically fluid), to be detected by “targets,” which are bound to the substrate at the various regions.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For example, in fluorescent detection embodiments, the scan region includes the entire area of the slide scanned in each pass of the lens, between the first feature of interest and the last feature of interest, even if there exist intervening areas that lack features of interest.

An “array layout” refers to one or more characteristics of the features, such as feature positioning on the substrate, one or more feature dimensions, and an indication of a moiety at a given location.

The assays of this disclosure are diagnostic and/or prognostic (predictive), i.e., diagnostic/prognostic. The term “diagnostic/prognostic” is herein defined to encompass the following processes either individually or cumulatively depending upon the clinical context: determining the predisposition to a disease, determining the nature of a disease, distinguishing one disease from another, forecasting as to the probable outcome of a disease state, determining the prospect as to recovery from a disease as indicated by the nature and symptoms of a case, monitoring the disease status of a patient, monitoring a patient for recurrence of disease, and/or determining the preferred therapeutic regimen for a patient. The diagnostic/prognostic methods of this disclosure are useful, for example, for screening populations for the presence of APKD, determining the risk of developing APKD, diagnosing the presence of APKD, monitoring the disease status of APKD, determining the severity of APKD, and/or determining the prognosis for the course of neoplastic disease.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids (e.g., surface bound and solution phase nucleic acids) of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the disclosure can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions that determine whether a nucleic acid is specifically hybridized to a surface bound nucleic acid. Wash conditions used to identify nucleic acids may include (e.g., a salt concentration of about 0.02 molar at pH 7 and a temperature of at least about 50° C. or about 55° C. to about 60° C.; or, a salt concentration of about 0.15 M NaCl at 72° C. for about 15 mins; or, a salt concentration of about 0.2×SSC at a temperature of at least about 50° C. or about 55° C. to about 60° C. for about 15 to about 20 mins; or, the hybridization complex is washed twice with a solution with a salt concentration of about 2×SSC containing 0.1% SDS at room temperature for 15 mins and then washed twice by 0.1×SSC containing 0.1% SDS at 68° C. for 15 mins; or, equivalent conditions). Stringent conditions for washing can also be (e.g., 0.2×SSC/0.1% SDS at 42° C.).

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “salts” herein refers to both salts of carboxyl groups and to acid addition salts of amino groups of the polypeptides of the present disclosure. Salts of a carboxyl group may be formed by methods known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the present disclosure or their analogs.

The term “polymorphism” as used herein refers to the occurrence of two or more genetically determined alternative sequences or alleles in a population. A polymorphic marker or site is the locus at which divergence occurs. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “genome” as used herein is all the genetic material in the chromosomes of an organism or host. DNA derived from the genetic material in the chromosomes of a particular organism is genomic DNA.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “T” at the polymorphic site, the altered allele can contain a “C”, “G” or “A” at the polymorphic site.

As used herein, the term “host” or “organism” includes humans, mammals (e.g., cats, dogs, horses, etc.), living cells, and other living organisms. A living organism can be as simple as, for example, a single eukaryotic cell or as complex as a mammal.

A “restriction enzyme” refers to an endonuclease (an enzyme that cleaves phosphodiester bonds within a polynucleotide chain) that cleaves DNA in response to a recognition site on the DNA. The recognition site (restriction site) may be a specific sequence of nucleotides typically about 4-8 nucleotides long.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present disclosure requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UITma, and variations and derivatives thereof.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±10%, or more of the numerical value(s) being modified. In addition, the phrase “about ‘x’ to ‘y’” includes “about ‘x’ to about ‘y’”.

Many variations and modifications may be made to the above-described embodiments and in the Appendices. All such modifications and variations are intended to be included herein within the scope of this disclosure.

### Discussion

Embodiments of the present disclosure encompass methods of isolating user-defined unique gene sequences from complex eukaryotic genomes. Embodiments of the present disclosure are advantageous because the total basecalling call rate has been determined to be greater than 99%. This very high level of coverage implies that embodiments of the present disclosure efficiently enrich for the variety of sequences contained in the genomic regions targeted. In addition, the reproducibility of RA base calls was about 99.98%. Furthermore, the accuracy at segregating sites was about 99.81%.

Embodiments of the method encompass shearing genomic DNA; repairing the genomic DNA fragments; hybridizing genomic DNA oligonucleotides of interest to a high density long oligonucleotide microarray; eluting of the genomic DNA fragments bound to oligonucleotides of interest on the microarray; and amplifying of eluted genomic DNA fragments. Additional details are provided in the Examples presented below.

The shearing of the genomic DNA may be conducted using physical shearing. In particular, the shearing of the genomic DNA can be conducted using sonication, nebulization or a combination thereof. The physical shearing is advantageous for at least the reason that it is a random process while other techniques, such as, but not limited to, enzymic cleavage are not completely random. The genomic DNA fragments are most advantageously from about 200 to 600 base pairs in length after shearing. The size of the genomic DNA fragments can be controlled by controlling the conditions of the solution and the conditions of the physical shearing such as, but not limited to, the duration or amount of applied energy. Further details are provided by the Examples below.

After shearing of the genomic DNA, the resulting genomic DNA fragments are end repaired. The genomic DNA fragments may be repaired using blunt end and phosphorylation reactions. Most advantageously, an adenosine (A) overhang or extension is added to the 3′ ends of the genomic DNA fragments. Next, the repaired genomic DNA fragments are ligated to the specifically designed adapters. The adapters prevent or reduce self ligation because of overhangs on each adapter, are unique relative to the target DNA genome, and are complimentary to one another. One example of an advantageous pair of complementary adaptor molecules have the sequences of SEQ ID NOs: 1 and 2. Further details are provided by the Examples below.

After the ligation reaction, the sample is cleaned and excess adaptors are removed. Subsequently, the genomic DNA fragments are hybridized to a high density long oligonucleotide microarray. In particular, the genomic DNA fragments are hybridized to a custom-designed high density long oligonucleotide microarray. In one embodiment of the disclosure, the custom-designed high density long oligonucleotide microarray may be generated by Nimblegen Systems Inc. (Madison, Wis.), wherein the array may include a plurality of unique oligonucleotide sequences of interest for each gene described above. Current Nimblegen Systems Inc. arrays can resequence about 45 kb to about 300 kb, depending upon the feature density. The genomic DNA fragments bound to oligonucleotides of interest on the microarray are then eluted. Further details are provided in Examples 3-13 below.

Next, the eluted genomic DNA fragments are amplified. In particular, the concentration of the eluted genomic DNA fragments is normalized for PCR amplification in multiple tubes, which significantly increases the efficiency of amplification, leading to better enrichment relative to other techniques. An advantageous amplification protocol for use in the methods of the disclosure is Ligation Mediated PCR (LPCR), as described in Example 12 herein.

The MGS protocol of this disclosure uses routine enzymatic reactions and protocols that increase efficiency while minimizing risk of contamination and artifacts. The capture arrays are standard high-density long oligonucleotide arrays and are commercially available. The user can design the array to select multiple unique sequence fragments located throughout the genome for resequencing, or to comprehensively resequence genomic regions without the repeat blocking step necessary for BAC genomic selection.

MGS, in addition to other general methods of multiplex amplification or sample enrichment (see for example Dahl. at al., Proc. Natl. Acad. Sci. U.S.A. 104, 9387-92 (2007) incorporated herein by reference in its entirety), has the advantage for laboratories with limited infrastructure and relatively few personnel, that they may be able to generate genome sequences at levels comparable to a conventional genome sequencing center. The ability of MGS to select multiple targets enables a comprehensive large-scale resequencing of user defined genomic regions that provide potentially important clues to the pathogenesis of complex diseases (Sjoblom et al., Science 314, 268-74 (2006)), or to find human genetic variation and functional sequences in both coding and non-coding regions (Dahl. et al., Proc. Natl. Acad. Sci. USA 104, 9387-92 (2007)). The methods of the disclosure may be advantageous for candidate gene studies that have been limited by sequencing capabilities and offers the opportunity to select hundreds of genes in known pathways for resequencing. MGS may also be advantageous in other eukaryotic model systems (i.e., mouse, zebrafish, Drosophila) to speed the sequencing of regions known to contain induced mutations.

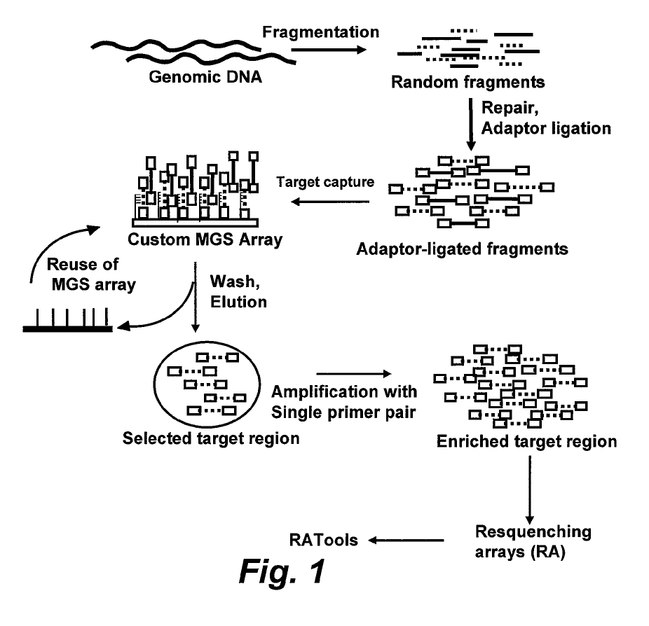

The present disclosure therefore encompasses methods (termed ‘Microarray-based Genomic Selection’ (MGS) capable of isolating user-defined unique genomic sequences from complex eukaryotic genomes. The MGS protocol of the disclosure encompasses five steps including, but not limited to, physical shearing of genomic DNA to create random fragments with an average size of 300 bp; end repairing of the fragments advantageously includes, but is not limited to, adding 3′-A overhangs, followed by ligation to unique adaptors with complementary T nucleotide overhangs; fragment hybridizing and capture using a custom high-density oligonucleotide microarray consisting of complementary sequences identified from a reference genome sequence; elution of fragments bound to the probes, and amplification of selected fragments through one round of PCR using adaptors as a single set of primers/template. FIG. 1 provides a schematic overview of one embodiment of the method, starting with genomic DNA and ending with finished sequence across the targeted regions. An exemplar protocol is outlined in the examples below.

The present disclosure, therefore, provides methods of isolating user-defined unique gene sequences from complex eukaryotic genomes comprising isolating genomic from a human or animal, shearing of the genomic DNA into fragments, repairing the genomic DNA fragments, ligating adapters to the genomic DNA fragments, hybridizing the genomic DNA fragments to oligonucleotides of interest of a high density long oligonucleotide microarray, eluting of the genomic DNA fragments bound to oligonucleotides of interest on the microarray; and amplifying the eluted DNA fragments.

In various embodiments of the disclosure, the methods may further comprise the resequencing of the eluted DNA fragments.

In an embodiment of the disclosure, the shearing is physical shearing. In some embodiments of the disclosure, the shearing is selected from sonication, nebulization, or a combination thereof.

In embodiments of the disclosure, repairing may include, but is not limited to, blunt end formation or the addition of 3′-A extensions to the genomic DNA fragments.

In one advantageous embodiment, repairing the genomic DNA fragments includes adding 3′-A extensions to the genomic DNA fragments.

In the embodiments of the disclosure, the adaptors may be blunt-end ligated to the genomic DNA fragments and the adapters may not substantially self ligate, are unique relative to the DNA genome, and are complimentary to one another.

In the embodiments of the disclosure, the adaptors may have a 3′-T extension and complement the 3′-A extensions of the repaired genomic fragments, and the adapters may not substantially self ligate, are unique relative to the DNA genome, and are complimentary to one another.

In an advantageous embodiment of the disclosure, the adaptors may have a 3′-T extension, and the adapters may not substantially self ligate, are unique relative to the DNA genome, and are complimentary to one another.

In one embodiment of the disclosure the adaptors may have the nucleotide sequences according to SEQ ID NOs: 1 and 2.

The present disclosure further provides an embodiment of a method of isolating user-defined unique gene sequences from complex eukaryotic genomes comprising, isolating genomic from a human or animal, shearing the genomic DNA into fragments, wherein the shearing is physical shearing selected from sonication, nebulization, or a combination thereof, repairing the genomic DNA fragment, wherein repairing includes using blunt end formation and phosphorylation reactions to repair the genomic DNA fragments, ligating a plurality of adapters to the genomic DNA fragments, wherein the adaptors are blunt-end ligated to the genomic DNA fragments, and wherein the adapters do not substantially self ligate, are unique relative to the DNA genome, and are complimentary to one another, and wherein the adaptors have the nucleotide sequences according to SEQ ID NOs: 1 and 2, hybridizing the genomic DNA fragments to oligonucleotides of interest of a high density long oligonucleotide microarray, eluting of the genomic DNA fragments bound to oligonucleotides of interest on the microarray; amplifying the eluted DNA fragments and resequencing of the eluted DNA fragments.

The following examples are provided to describe and illustrate, but not limit, the claimed disclosure. Those of skill in the art will readily recognize a variety of non-critical parameters that could be changed or modified to yield essentially similar results.

### EXAMPLES

### Example 1

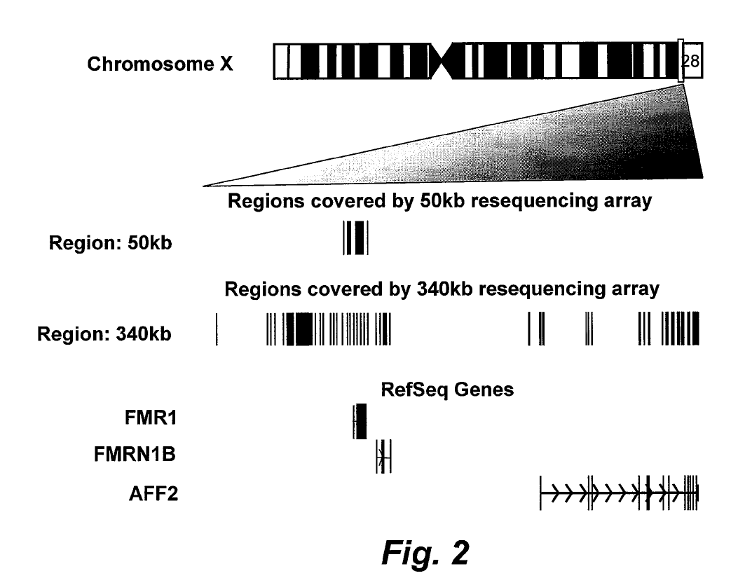

Two X-linked genomic regions were captured and resequenced, as shown in FIG. 2. The initial experiment examined a region 50 Kb in size that included coding and non-coding sequences surrounding the fragile X mental retardation gene (FMR1). In a second, larger scale experiment, 304 Kb of unique coding and non-coding sequences contained within a 1.7 MB genomic region that includes FMR1, FMR1NB and the AFF2 genes was isolated and resequenced. Each custom MGS array consisted of approximately 385,000 long oligonucleotide capture probes (each typically being between 50 bp and 93 bp) covering the regions of interest.

The oligonucleotide probes were manufactured by NimbleGen Systems, Inc. (Madison, Wis.). Capture probe sequences included both the forward and reverse strands manufactured on a standard commercially available microarray according to the specifications given in Example 2 below. For the 50 Kb region, there were four pairs of probes for every targeted base, while the 304 Kb region had one pair of probes for every 1.5 targeted bases. The capture oligonucleotides were between 50 and 93 base pairs long and were designed to achieve optimal isothermal hybridization across the microarray.

Twenty micrograms of whole genome amplified genomic DNA were processed for each sample using the MGS protocol. Upon eluting the selected target from the capture MGS chip, yields of between 700 ng and 1.2 μg were obtained. The eluted sample was split into between 5 and 10 PCRs, each of which was carried out using high fidelity Taq polymerase at an optimal concentration of 3 ng/μl of PCR template. MGS capture chips could be used at least one time with no apparent contamination or effect on data quality (data not shown).

### Example 2

Assessment of the MGS: To assess MGS, a 50 kb genomic region containing the FMR1 locus in cell lines derived from 2 patients with known FMR1 mutations was resequenced: FMR1 mutation Tr91 contains a disease causing point mutation (A>T) at position 146825745 on the X chromosome while DM316 harbors a large deletion of the FMR1 gene (De Boulle et al., Nat Genet 3, 31-5 (1993); Gu et al., Hum Mol Genet 3, 1705-6 (1994).

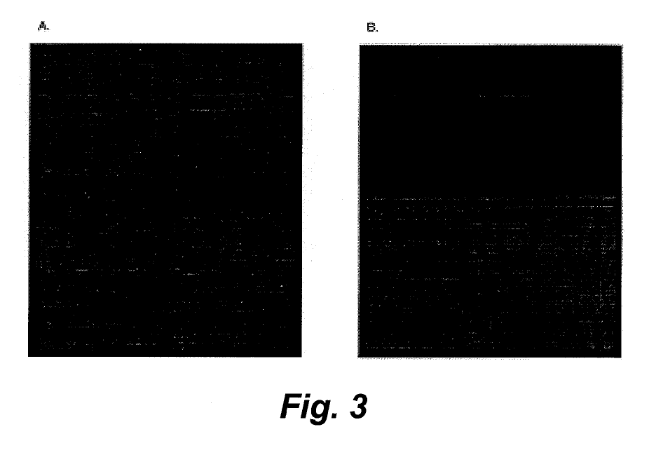

A NimbleGen 50 Kb resequencing array was designed that covered the targeted regions, containing both coding and non-coding sequences in the vicinity of the FMR1 gene (as shown in FIG. 2), and resequenced both patients in triplicate using MGS (see Example 3). Analysis of the TR91 sequence identified the expected A>T point mutation when compared to the human genome reference sequence in all three replicates. Six additional variants were detected in TR91, 5 of which were successfully validated by independent sequencing. Each of the three DM316 samples exhibited an absence of hybridization on the resequencing array (RA) in the regions corresponding to the known deleted sequences, as shown in FIG. 3.

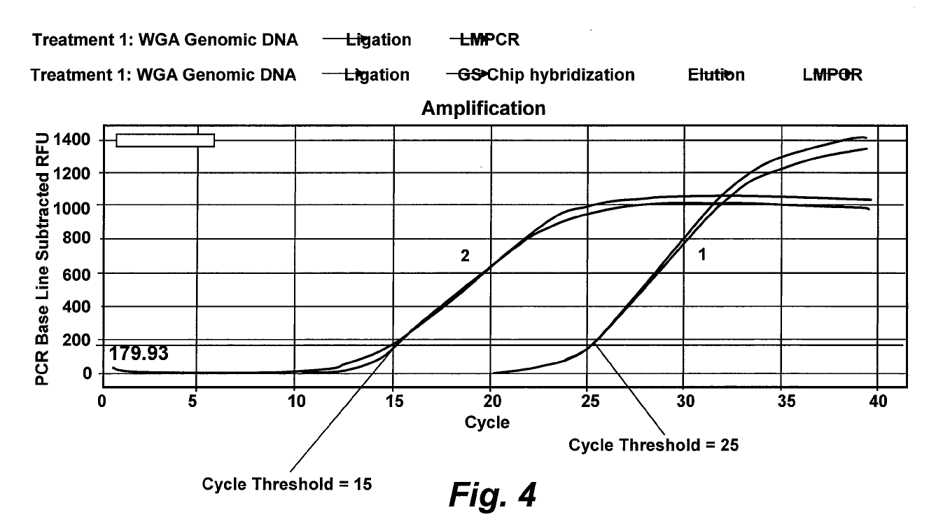

To evaluate MGS on a larger genomic region, a total of 304 Kb was selected from 10 individual genomes represented by two populations of different ancestry: a European descent (ED) population (n=5) selected from the Centre d'Etude du Polymorphism Humain (CEPH) panel and an African descent (AD) population (n=5) selected from the Hapmap (Coriell Cell Repository numbers provided in Supplementary Methods). MGS was replicated twice for each of the ten samples. Using quantitative PCR, it was estimated that MGS enriched targeted sequences approximately 1000-fold, as shown in FIG. 4.

The resequencing results provided three lines of evidence demonstrating the efficacy of the MGS protocol. First, our total basecalling call rate over all 20 replicates (10 samples each processed twice) was about 99.1% (6,528,393 called out of 6,585,832 total), implying that MGS protocol efficiently enriches for the variety of sequences contained in the genomic regions we targeted. Second, for each sample, we counted the number of bases called identically and differently between both replicates. The reproducibility of RA base calls was about 99.98%. Third, for each sample, to assess accuracy of basecalls, the RA basecalls with genotype calls generated by the HapMap project were compared. There were 39 discrepancies between RA and HapMap genotype calls. To identify the nature of the discrepancy, each was independently resequenced via conventional ABI chemistry. The resulting sequence data showed that 27 of the discrepancies agreed with our RA call, while 12 agreed with the HapMap genotype call. Hence, more than two thirds of the discrepancies observed arose due to errors in HapMap genotyping. The final accuracy at segregating sites was thus about 99.81%.

### Example 3

Array Design: The UCSC Table Browser function with repeats masked on the latest human genome build (March 2006) were used to identify the unique sequences within a selected genomic region (Karolchik et al., Nucleic Acids Res 31, 51-4 (2003)). The CGG repeat sequence of FMR1 from the human genome reference sequence was included in the design. Since genetic variants in regulatory elements away from the coding sequences may influence the expression of a gene, unique sequences upstream and downstream of the target genes were also included. These unique sequence were then screened to obtain approximately 50 Kb or 304 Kb of unique sequence. Unique sequences 100 by or less were ignored and in some cases, short (<100 bp) stretches of previously masked sequence were included to avoid breaking up long stretches of genomic regions.

The FASTA format sequences were then provided to chip design engineers at Nimbelgen (Madison, Wis.) to select oligonucleotides for the microarray-based genomic selection (MGS) array. Standard bioinformatics filters that check for genomic uniqueness against an indexed human genome (15mers) were used to select capture oligos. The capture oligonucleotides were between 50 and 93 basepairs long and were designed to achieve optimal isothermal hybridization across the microarray. No other optimization of oligos was performed. For the 50 kb region, there were four pairs of probes for every targeted base, while the 300 kb region had one pair of probes for every 1.5 targeted bases.

Resequencing arrays: Resequencing arrays were designed from the FASTA format sequences provided to design engineers at Affymetrix (Santa Clara, Calif.) (FMR1/FMR2) and NimbleGen (Madison, Wis.) (FMR1 only).

Resequencing Arrays (RAs) query a given base by using overlapping oligonucleotide probes, tiled at a 1-basepair (bp) resolution. The oligonucleotide probes, referred to as features, are typically 25 by long. Both the forward and reverse strands are interrogated, so sequencing a single base requires a total of 8 features. A set of four features contains oligonucleotides identical to the forward reference strand, except at position 13 (the base to be queried), where there is either A, C, G, or T. The remaining four features are similarly designed for the complementary strand. When a labeled DNA sample, called a target, is hybridized to these eight features on the array, the two features complementary to the reference sequence (forward and reverse complement) will yield the highest signal. If, however, the target DNA contains a variant base at position 13, the two features complementary to that variant base will yield the highest signal. Given eight features for each base, interrogation of an L-length duplex strand would require 8 L oligonucleotide probes.

### Example 4

Sample Selection: DNA samples were purchased from the Coriell Cell Repository (Camden, N.J.) and included 10 individual genomes represented by two populations of different ancestry: a European descent (ED) population (n=5) selected from the Centre d'Etude du Polymorphism Humain (CEPH) panel with the Coriell Cell Repository numbers: NA07029, NA07048, NA10846, NA10851 and NA10860; and an African descent (AD) population (n=5) selected from the Hapmap with the Coriell Cell Repository numbers: NA18500, NA18503, NA18506, NA18515 and NA18521. MGS was replicated twice for each of the ten samples. Other samples were extracted from cell lines representing fragile X patients with either disease-causing point mutation (A>T) at position 146825745 on the X chromosome (Tr91) or a deletion (DM316) in the fragile X mental retardation (FMR1) gene (Boulle et al. Nat Genet 3, 31-5 (1993); Gu et al., Hum Mol Genet 3, 1705-6 (1994)). Primer sequences used in independent sequencing validation of HapMap and Tr91 discrepancies are given in Table 1.

TABLE I

| | | SEQ ID |
|---|---|---|
| | | NO |
| HAPMAP SAMPLES | | |
| rs16994908_FW2 | CTTCACCATTTTTGCATGTACC | 3 |
| rs16994908_REV | TTGCAACCACATTTGAAGTGAC | 4 |
| rs12688573_FW | AAAGTCGCACAGATACCCTCTC | 5 |
| rs12688573_REV | CTTTTCTGTCTTGCCATTAGCC | 6 |
| rs11117557_3_FW | ACTGCATCTGCAGAGAAACAAC | 7 |
| rs11117557_3_REV | AACAGTTGTGAAACTACGTCAGG | 8 |
| rs7052829_FW | TTATGGGAAGAATCCACTCCAG | 9 |
| rs7052829_REV_2 | AGTAGCAGCAACAGCAACAAAG | 10 |
| rs7052654_rpt_FW | CAGGGCAGGGATGATTAGAG | 11 |
| rs7052654_rpt_REV | AGAAAGGAAGAGATGCATGGAC | 12 |
| rs6626955_6_FW | TCCCTTGTGTTCATGGAGTATG | 13 |
| rs6626955_6_REV | AACAGGAGCTTCTTCCTGATTG | 14 |
| rs2761622_2_FW | AAATGAAATGCACCTTCCAGAG | 15 |
| rs2761622_2_REV | GCACTTGTTTCACAGGTACAGC | 16 |
| rs1805422_FW | GTAGCAGTAGTGCGTTTGTTGG | 17 |
| rs1805422_REV | TTTCCTATAGCCAAACGTGTCC | 18 |
| rs1265401_FW | GGGTATGGGTTTAACATAGGACAG | 19 |
| rs1265401_REV | GACTTACGGGCTGCTTCTCAC | 20 |
| rs1265397_FW | GCATGCGTGTCTTACTCCATAG | 21 |
| rs1265397_REV | AAGCTCTGTCAGTGTGATGTGG | 22 |
| rs25699_FWD | GCCAGAGGCTATTTCCCTAACTTAC | 23 |
| rs25699_REV | TGATGACGAACTCTGGAATTTGAC | 24 |
| rs4949_FWD | AGAGTGCTTTTGTTGGGATGTAC | 25 |
| rs4949_REV_2 | ATTACACACATAGGTGGCACTA | 26 |
| rs1442280_FWD | AGACATTGCAAACATCCAGAAC | 27 |
| rs1442280_REV | ATGCAGTCAGCCAGGTAATAGA | 28 |
| rs16994869_FWD | TGAACAGTCACTTGACATCCAAAG | 29 |
| rs16994869_REV | GATTGGAGGAGGCAGAGAAATAGT | 30 |
| Tr91 | | |
| rs29284_int9_FW | CTCTGGTACCTGACCAAAGGAG | 31 |
| rs29284_int9_REV | AAAGCAGTAAGCACAGCCCTAC | 32 |
| rs29288_int13_FW | CATGCCATTCATTCTTATGGTG | 33 |
| rs29288_int13_REV | AATCCTAACTCTCCAGGCCTTC | 34 |
| rs25707_ex5_FW | CCTGCCACAAAAGATACTTTCC | 35 |
| rs25707_ex5_REV | TTCTGCATTGCTCTTGCAAAC | 36 |
| I304N_ex10_FW | ACAGTAGGGCTGTGCTTACTGC | 37 |
| I304N_ex10_REV | CTCATTTTCAGCCTCAATCCTC | 38 |
| rs29286_int12_FW | GTGGCTTCATCAGTTGTAGCAG | 39 |
| rs29286_int12_REV | CACATACCCACAAACACTCCTC | 40 |
| rs5904816_int14_FW | GCACATCAAGGTTTGAACTTAGG | 41 |
| rs5904816_int14_REV | CAGAGACGTTTCAGGGGTAATC | 42 |
| rs25704_ex17_FW | GGAAGGTCATTTCCATGTATGC | 43 |
| rs25704_ex17_REV | AAAACCAAACCCCAACACTTC | 44 |

Genomic DNA should be assessed for integrity and purity. A 1.0% TAE gel is run and the DNA quantified by Nanodrop. The A260/280 ratio should be >1.8

### Example 5

Adaptor and Primer Design: All oligonucleotides used were obtained from Invitrogen Corp (Carlsbad, Calif.). The adaptor was prepared by annealing the forward (21 bp) and reverse (22 bp) oligonucleotides to generate a 21 by dsDNA fragment with single and double base “T” overhangs at the 3 prime and 5 prime end respectively. Adaptor sequences used were 5′-CTCGAGAATTCTGGATCCTCT-3′ (SEQ ID NO: 1) and 5′-TTGAGCTCTTAAGACCTAGGAG-3′ (SEQ ID NO: 2). Annealing of the oligos was performed by mixing both oligonucleotide to a final concentration of 1.5 μg/μl of each oligonucleotide, heating to 95′C for 10 mins in a heating block, turning off the heating block and allowing the mixture to slowly cool back to room temperature. The primers used for the enrichment were made by preparing a 20 μM of each oligonucleotide used for the adaptor.

### Example 6

Genomic DNA preparation: Whole genome amplification was performed on 250 ng of genomic DNA using the Repli-g MIDI™ Kit (Qiagen Inc., Valencia, Calif.). Following amplification, the unpurified samples were quantified using a spectrophotometer (NanoDrop, Wilmington, Del.). Twenty-five micrograms of each sample was aliquoted into sterile Eppendorf tubes for a final concentration of 100 ng/μl (250 μl).

### Example 7

Target DNA isolation: Samples were sonicated (Misonix sonicator 3000, Misonix, Farmingdale, N.Y.) in eppendorf tubes with a microtip probe using the following parameters: 3 pulses of 30 seconds each with 2 mins of rest and a power output level of two. After fragmentation, approximately 300-500 ng of each sample was run on a 1.0% TAE agarose gel against 300-500 ng of a 1 Kb plus ladder to verify that fragments average 300 by in size. The remaining samples were then dried down in a SpeedVac at medium heat to 55 μl (75° C.).

### Example 8

Repairing Ends of Sheared DNA and 3′ tail addition: To the 25-30 μg fragmented DNA were added 10 μl of dNTPs (2.5 mM, TaKaRa), 10 μl of 10×T4 DNA Polymerase Buffer (NEB), 1 μl of 100×BSA (NEB), 15 μl of T4 DNA Polymerase (3 U/μl, NEB). The mix was then incubated in a thermocycler at 12° C. for 20 mins, and 70° C. for 5 mins followed by 37° C. for 30 mins.

After incubation 2 μl of 10×T4 DNA Polymerase Buffer (NEB), 1 μl 100 mM dATP (Sigma), 3 μl of 50 mM MgCl2, and 5 μl of Taq DNA Polymerase (5 U/μl, NEB) were directly added. Samples were incubated in a thermocycler at 72° C. for 35 mins.

After incubation the Promega Wizard® SV Gel and PCR Clean-Up Systems were used following the manufacturer protocols. Each column was eluted with 80 μl of water, the volume adjusted to 70 μl and 1 μl removed to perform Nanodrop quantification. The percent recovery should be consistently greater than 80% (20 μg) of the starting amount. The protocol is not continued unless this is the case.

### Example 9

Ligation of Adapters: The number of ends available for ligation in pmoles could be calculated as follows:

pmol ends/μg of DNA=(2×10⁶)/(number of base pairs×660)

The ratio of adapter to DNA should be at least about 12:1. While this increased the chance of getting some adapter concatamer (which should not hybridize to the array), all of the fragments would likely get adapters, which is very important. The following ligation reaction is based on using 25 μg of DNA (300 by average size). The amount of adaptor must be adjusted to maintain the ratio. The ligation reaction(s) were performed in a 0.2 ml PCR tube. To the 70 μl repaired reaction 10 μl of 10×T4 DNA Ligase Buffer (NEB) (kept on ice at all times), 15 μl of Adapters (1.5 μg/μl) and 5 μl of T4 DNA Ligase (2000 U/μl, NEB) was added. This was incubated at room temperature for 2 hours. The insert to vector ratio was calculated in terms of insert ends to vector ends.

When the ligation was complete, the sample was transferred to a 1.5 ml tube and 100 μl of VWR water was added. The Promega Wizard® SV Gel and PCR Clean-Up System was used following the manufacturer protocols. Each column was eluted with 50 μl of water and 1 μl was removed to perform Nanodrop quantification. The percent recovery should be consistently greater than 80% (20 μg) of the starting amount. The protocol is not continued unless this is the case.

### Example 10

Hybridization: To the ligated sample (15 μg) were added a 5-fold amount (in μg) of human Cot-1 DNA (Invitrogen). The sample was dried in the Speed-Vac at medium heat (75° C.) for 45 mins. The sample was vortexed for 3 mins and drying continued to the pellet.

The following reactions were performed in a 1.5 ml tube. To the pellet from dried sample 7.2 μl of VWR water, 8.25 μl of 2× Hybe Buffer (NimbleGen) and 1.43 μl Hybe Component A (NimbleGen) was added. The samples were vortexed 3 mins and then heated at 95° C. for 10 mins. The samples were quickly spun down and placed in the MAUI heat block at 42° C. until ready to use. Once the samples were applied to the chip surface, the mixer was begun on program B and hybridized for 60 hours.

### Example 11

Elution: Buffers are prepared about 30 mins prior to starting to allow the two stringent buffers to come to temperature. DTT is added immediately before use to minimize oxidation. The wash bin of wash 1 should be at 42° C. when it is used Volumes of buffers to prepare are shown in Tables 1 and 2.

TABLE 1
Buffer preparation for 1-2 samples

| | 10x | | | | | | | | 2x | | 2x | |
|---|---|---|---|---|---|---|---|---|---|---|---|---|
| | wash I | | 10x | | 10x | | 10x | | stringent | | stringet | |
| | (bin) | | wash I | | wash II | | wash III | | wash | | wash | |
| VWR water | 225 | ml | 25 ml from | | 22.5 | ml | 22.5 | ml | 12.5 | ml | 12.5 | ml |
| Wash | 25 | ml | wash bin I | | 2.5 | ml | 2.5 | ml | 12.5 | ml | 12.5 | ml |
| 1 M DTT | 25 | μl | | | 2.5 | μl | 2.5 | μl | 2.5 | μl | 2.5 | μl |
| Total | 250 | ml | | | 25 | ml | 25 | ml | 25 | ml | 25 | ml |
| VWR water | 225 | ml | 225 | ml | 225 | ml | 225 | ml | 125 | ml | 125 | ml |
| Wash | 25 | ml | 25 | ml | 25 | ml | 25 | ml | 125 | ml | 125 | ml |
| 1 M DTT | 25 | μl | 25 | μl | 25 | μl | 25 | μl | 25 | μl | 25 | μl |
| Total | 250 | ml | 250 | ml | 250 | ml | 250 | ml | 250 | ml | 250 | ml |

After hybridization, the MGS arrays were first prewashed at 42° C. in NimbleGen Buffer 1 followed by two 5 min washes at 47.5° C. with Nimblegen Stringent Buffer. The arrays were then washed at room temperature for 2 min with NimbleGen Buffer 1, 1 min with NimbleGen Buffer 2 and 30 seconds with NimbleGen Buffer 3. The washed chip was placed on the Hybriwheel (NimbleGen) at 100° C. and secured with a Hybe Puck (NimbleGen). 400 μl of 95° C. VWR water were added and incubated 5 mins. After the 5 mins incubation as much water as possible was removed and pipetted it into a labeled 1.5 centrifuge tube (placed on ice). This process was repeated one more time beginning with the addition of 400 μl of 95° C. VWR water to the puck. When this was complete, 350-400 μl of 95° C. VWR water was added and removed immediately and pipetted it into the 1.5 ml tube.

After elution, the sample was placed in the Speed-Vac at medium heat (75° C.) for 45 mins. The sample was vortexed for 3 mins and drying continued until the sample was to the pellet. The pellet was hydrated in 33 μl of VWR water and vortexed for 3 mins and Nanodrop quantification of single strand DNA (DNA-33) was used to determine the concentration of the sample (picogreen and ethidium bromide quantification are inefficient for single stranded DNA). Upon eluting the selected target from the capture MGS chip, yields of between 700 ng and 1.2 μg were obtained.

### Example 12

Amplification by Ligation Mediated PCR (LMPCR): Each eluted sample was amplified using a single primer pair represented by the adaptors oligos and a high fidelity polymerase. To maintain an optimal concentration of 3 ng/μl of template for each 50 μl PCR reaction, between 5 and 10 PCR reactions were done to amplify each entire eluate. One 50 μl reaction included 5 μl of 10×LA PCR buffer (TaKaRa), 5 μl of 2.5 mM dNTPs mix (TaKaRa), 2 μl of 20 μM FWD LMPCR primer, 2 μl of 20 μM REV LMPCR primer, and 2 μl of LA Taq (5 U/μl, TaKaRa), and VWR water to 50 μl volume. The reactions were incubated in a thermocycler at (1) 95° C. for 2 mins, (2) 95° C. for 60 seconds, (3) 58° C. for 60 seconds, (4) 72° C. for 60 seconds, (5) Repeat step 2 30 times (35 cycles), then at 72° C. for 5 mins and finally hold at 4° C.

All PCR reactions were pooled by sample and transferred into a 1.5 ml tube. Promega Wizard® SV Gel and PCR Clean-Up Systems were used following the manufacturer centrifugation protocol to purify the sample. For spin steps we used 13,000 g, and for the elution spin we used 16,000 g and 1.5 mins. Each column was eluted with 50 μl of water.

Three to 5 μl were used to verify size distribution on 1.5% TAE agarose gel against 500-750 ng of 1 Kb plus ladder and positive control (6× xylene cyanol loading dye for samples). Then the samples were quantified using Nanodrop and sonicated.

### Example 13

Resequencing of Selected DNA: NimbleGen's Comparative Genomic Sequencing protocol was used for the 50K RA. Briefly, 1 μg of sample was denatured at 98° C. for 10 mins in random primer buffer and labeled in the dark with Cy3-9mer primers (TriLink BioTechnologies, San Diego, Calif.) in the presence of dNTP mix and 100 units of Klenow (50 U/μl, NEB) for 2 hours. To guarantee at least 20 μg of label sample for resequencing, 2 labeling reactions were done per sample (2 μg total). Labeled samples were purified using ethanol precipitation method and dried down to the pellet in the dark to avoid bleaching of the Cy3 dye. After rehydrating the pellets with 20 μl total of VWR H₂O, ten to thirty micrograms of labeled DNA was mixed with NimbleGen's Hybridization cocktail (2× Hybe buffer and Hybe component A) and denatured at 95° C. for 5 min. The arrays were loaded and incubated overnight at 42° C. on MAUI Hybridization System (BioMicro). The signal was detected by measuring Cy3-chrome fluorescence using Genepix 4000B (Molecular Devices Corp., Sunnyvale, Calif.).

For Affymetrix RAs, 30 μg of enriched samples were digested to 20 to 100 by for 3 mins in a 42 μl reaction comprised of 10× Phor-All_Buffer (Amersham Biosciences), 10× Acetylated BSA and 3 units of DNAse1 (Promega). Reactions were heated at 75° C. for 10 mins to inactivate the DNAse then to 95° C. for 15 mins to separate the strands. The reactions were then cooled at 4° C. for 45 mins. The fragmented DNA was labeled using 17.13 nmol of a biotinylated proprietary labeling reagent (Affymetrix), 4.5 units of terminal deoxynucleotidyl transferase (Affymetrix) and terminal deoxynucleotidyl transferase buffer (Affymetrix) at a final concentration of 1×. The reactions were brought to a volume of 60 μl with nuclease free water (VWR). Each reaction was incubated at 37° C. for 4 hours followed by heat-inactivation for 15 mins at 95° C. and stored at 4° C. until ready to use.

The labeled DNA samples were combined with 160 μl hybridization buffer comprised of 1M Tris HCl pH 7.8 (Sigma), 5M TMACL (Sigma), 0.10% Tween 20 (Pierce Biotechnology), 100 μg/μl of herring sperm DNA (Promega), 500 ug/ml Acetylated BSA (Invitrogen), and 200 pM biotinylated SNPHy948B (Invitrogen). The hybridization mix was then heated to 95° C. for 5 mins, equilibrated at 49° C. and hybridized to the high-density oligonucleotide array at 49° C. for 16 hours. All signal detection steps were performed using an Affymetrix fluidics.

The arrays were washed in 6×SSPE, 0.01% Tween 20 solution (wash A) 6 times at 25° C. then in 0.6×SSPE, 0.01% Tween 20 solution (wash B) 6 times at 45° C. For signal detection, the arrays were incubated with stain 1 (6×SSPE, 0.01% Tween 20, 1×Denhardt's solution (Sigma), and 10 ug/ml SAPE (Invitrogen), final concentration) for 10 mins at 25° C., followed by 6 washes with wash A at 25° C. Incubation with stain 2 (6×SSPE, 0.01% Tween 20, 1×Denhardt's solution (Sigma), and 10 ug/ml anti-streptavidin antibody final concentration was done for 10 mins at 25° C. A second incubation with stain 1 was done for 10 mins at 25°. The arrays were rewashed 10 times in wash A at 30° C. and filled with a holding buffer (5M NaCl, 10% Tween 20, MES hydrate and MES sodium salt). They were stored at 25° C. until they were ready to be scanned. The signal was detected by measuring Cy-chrome fluorescence using a G7 Genechip scanner (Affymetrix). For both the Nimblegen and Affymetrix resequencing arrays, all bases calls were made with the RATools program RA_PopGenCaller

### Example 14

Validation Sequencing: Discrepancies between RA data and HapMap data were evaluated using independent sequencing. PCR primers were designed using Primer 3 software. PCR Reactions were composed of 400 ng of sample DNA was mixed with 8 μl of dNTP mix (TaKaRa), 5 μl of 10×LA Taq buffer (TaKaRa), 1.5 μl LA Taq (TaKaRa), 0.8 μl of each forward and reverse primers and VWR water to 50 μl total reaction volume. DNA was amplified using the following parameters: 94° C. for 4 min, 30 cycles of 94° C. for 20 sec, 58° C. for 1 min, and 72° C. followed by 72° C. for 5 mins. This method was also used to validate discrepancies in the Tr91 RA data. The primers that amplified the SNP discrepancies are listed in Table 1, Example 5.

PCR products were run on a 1% TAE agarose gel, excised from the gel and purified using the Promega Wizard® SV Gel and PCR Clean-Up System.

### Example 15

Long PCR Control: To minimize the number of amplifications, we used long PCR to amplify genomic regions that contain one or more unique sequence blocks tiled onto the variant resequencing array. A total of 14 primer pairs spanning 48 Kb (including the 39 kb FMR1 genome region) were used. Except for one primer close to the CGG repeat (20 bp), Long PCR primers were 31 to 34 base pairs long and were selected by using Amplify 3.1.4 to ensure that they bound uniquely within a 48 kb region and had a primer stability value between 70 and 80. Primers had GC content between 45% and 60%.

Amplification of genomic DNA was accomplished in 50 μl reactions carried out in thin-walled polypropylene tubes using LA Taq (TaKaRa). The manufacturer's recommendation was followed. LPCR amplification of the human samples employed either a standard or a modified mixture where 5% DMSO (or manufacturer GC Buffer) was added to aid the amplification of GC rich regions. The standard conditions for the LPCR were: (1) 94° C. for 2 mins, (2) 94° C. for 10 seconds, (3) 68° C. for 1 minute per kb fragment size, (4) repeat to step 2, 30 times, and (5) final extension time equal to step 3 plus five mins. Each LPCR required a minimum of 200 ng of human genomic DNA and most fragments were between 3.4 and 11 kb long. To obtain optimal performance across the microarray, equal molar concentration of PCR product were pooled, to ensure that an equal number of targets existed for each probe on the array.

### Example 16

Quantitative PCR: Quantitative PCR was performed on sample DNA with two treatments: (1) whole genome amplified, ligated and then amplified using LMPCR protocol but never hybridized to a genomic selection array (Treatment 1) and a (2) whole genome amplified, ligated, hybridized to a genomic selection array, eluted from the array, and then amplified using LMPCR. Reagents used included iQ SYBR® Green Supermix (Bio-Rad, Hercules, Calif.) and the following primer pair:

| | | |
|---|---|---|
| FW: | 5′-ACAGTAGGGCTGTGCTTACTGC-3′ | (SEQ ID NO: 1) |
| REV: | 5′-CTCATTTTCAGCCTCAATCCTC-3′ | (SEQ ID NO: 2) |

The primers amplify 156 bases from exon 10 in the FMR1 gene. Reactions contained 12.5 μl of 1×iQ SYBR® Green Supermix, 1 μl of FW Primer (10 mM), 1 μl of REV Primer (10 mM), 9.5 μl of VWR water and 1 μl of DNA template (30 ng/μl) for a total volume of 25 μl. The standard curve was created using whole genome amplified DNA at concentrations ranging from 7.8 ng/μl to 500 ng/μl. The reactions were performed in triplicate. The reactions were incubated in a Bio-Rad iQ5 Multicolor Real Time PCR Detection Light Cycler using the following parameters: (1) 94° C. for 3 mins, (2) 94° C. for 10 seconds, (3) 58° C. for 30 seconds, (4) 72° C. for 30 seconds, and (5) Repeat steps 2-4 for 40 cycles.

From the quantitative PCR result it was conservatively estimated that at least 1000× enrichment of DNA used for resequencing (treatment 2) when compared to whole genome amplified DNA that underwent LMPCR amplification (treatment 1). The DNA from treatment 2 had a cycle threshold of 15 while the cycle threshold for treatment 1 was 25. Assuming that DNA concentration doubles every cycle, then enrichment can be calculated by 2^{N}, with N equaling the difference between the cycle thresholds of the two treatments (see FIG. 4).

### Example 17

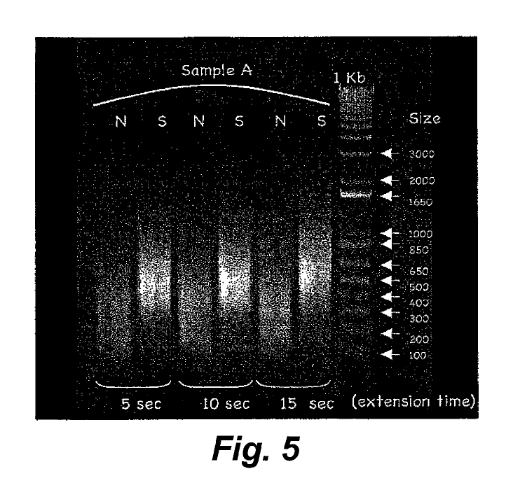

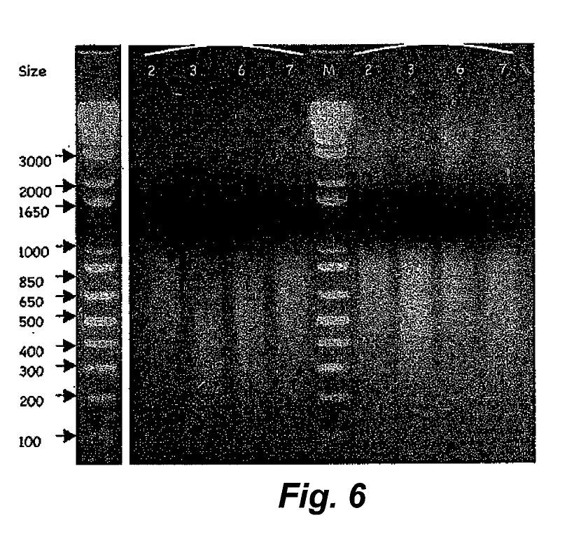

For genomic DNA fragmentation on a BAC (RP11-489K19), sonication performed better than nebulization, as shown in FIG. 5. The second goal was to test our target DNA production protocol in DNA from a BAC (RP11-489K19) containing the region of interest, a variety of dilutions of that BAC with other non-specific BACs, and finally human genomic DNA from a normal and a patient with a point mutation. The results are presented in Table 2 and FIG. 6). The percent of bases called with DNA derived from the BACs was excellent. The human genome sample results (47.4%) were lower than we desired, but we believe that improving the PCR amplification and increasing the quantity of DNA hybridized to the array will substantially improve this value. Experimental analysis of the data is continuing to further characterize the nature of the chip resequencing data.

TABLE 2

| | Dilution | | Processing | % Base |
|---|---|---|---|---|
| Samples | ratio | Description | status | called |
| A | None | FMR1 BAC49K19 | Completed | 99% |
| | | genomic DNA | Mar. 03, 2006 | |
| 1 | None | FMR1 BAC49K19 | In progress | |
| | | genomic DNA | | |
| 2 | 1 11.5 | FMR1 BAC49K19/mixof | Completed | 98.9% |
| | | unrelated BAC DNA | Jun. 08, 2006 | |
| 3 | 1/15 | FMR1 BAC49K19/mixof | Completed | 97.9% |
| | | unrelated BAC DNA | Jun. 08, 2006 | |
| 4 | 1/30 | FMR1 BAC49K19/mixof | In progress | |
| | | unrelated BAC DNA | | |
| 5 | 1/60 | FMR1 BAC49K19/mixof | In progress | |
| | | unrelated BAC DNA | | |
| 6 | None | FMR1 BAC49K19 | Completed | 99.2% |
| | | genomic DNA | Jun. 08, 2006 | |
| 7 | None | Normal Human | Completed | 47.4% |
| | | Genomic DNA | Jun. 08, 2006 | |
| 8 | None | FMR1 point mutation | In progress | |
| | | Human genomic DNA | | |

TABLE 3
Resequencing Results after Genomic Sequencing

| | % Conformity | |
|---|---|---|
| | (Calculated with | % Basecalling |
| Sample (date) | Nimblescan) | (RATools-ABACUS) |
| Tr91 (Jan. 14, 2007) | 99.28% | 98.0% |
| J1 (Jan. 21, 2007) | 98.65% | 91.8% |
| Tr91 (Jan. 21, 2007) | 98.93% | 97.8% |
| J1 (Jan. 23, 2007) | 99.09% | 92.3% |
| Tr91 (Jan. 23, 2007) | 99.3% | 98.3% |

ABACUS Parameters Used:
Quality Score Threshold of 30
Strand Threshold −2

Previous data demonstrates that these thresholds correspond to phred 56 (less than 1 error per 398,452 bases independently sequenced)

### Example 18

Initial Analysis and Comments on Resequencing Data Quality. The data archive listed above contains the resequencing data results from 3 initial TR91 chips. The genomic selection protocol was performed independently three times. The resulting fragments were then labeled and hybridized to a custom designed Nimblegen resequencing array (RA) for resequencing 48 kb from the FMR1 genomic region.

The RAs were analyzed with RATools (an open source implementation of the ABACUS algorithm). They were run at the following parameters:
“Total Threshold”, 30
“Strand Threshold”, −2
“Maximum percentage of N's before base is N'd out in all individuals”, 0.5
“Maximum percentage of N's before an entire Fragment is N'd out”, 0.5
“Window size for neighborhood rule”, 21

Fifteen chips were analyzed (chips were scanned multiple times at different photomultiplier tube—PMT values)—these chips were derived from 5 independent experiments, 3 of which used the TR91 cell line. The best three TR RAs were selected for analysis. They all called more than 97% of bases.

Analysis of all three chips against each other observed 7 discrepancies out of 140,999 total comparisons. This corresponded to a discrepancy rate of 4.96E-05 and a phred score of 43.0. This value of data quality exceeds the Bermuda standard and suggests high data quality in a single experiment. Typical genome sequences only achieve very high quality scores after performing multiple sequence reads. Furthermore, these results indicated that the genomic selection protocol is not inducing large numbers of new mutations. This Taq has a built-in proof reading exonuclease activity and thus must act to minimize mutations induced during the process of genomic selection.

1. A method of isolating user-defined unique gene sequences from complex eukaryotic genomes comprising:
isolating genomic from a human or animal;
shearing the genomic DNA into fragments;
repairing the genomic DNA fragments;
ligating adapters to the genomic DNA fragments;
hybridizing the genomic DNA fragments to oligonucleotides of interest of a high density long oligonucleotide microarray;
eluting of the genomic DNA fragments bound to oligonucleotides of interest on the microarray; and
amplifying the eluted DNA fragments.

2. The method of claim 1, further comprising resequencing of the eluted DNA fragments.

3. The method of claim 1, wherein the shearing is physical shearing.

4. The method of claim 3, wherein the shearing is selected from sonication, nebulization, or a combination thereof.

5. The method of claim 1, wherein repairing includes using blunt end formation or the addition of 3′-A extensions to the genomic DNA fragments.

6. The method of claim 1, wherein repairing the genomic DNA fragments includes the addition of 3′-A extensions to the genomic DNA fragments.

7. The method of claim 1, wherein the adapters do not substantially self ligate, are unique relative to the DNA genome, and are complimentary to one another.

8. The method of claim 1, wherein the adaptors have the nucleotide sequences according to SEQ ID NOs: 1 and 2.

9. A method of isolating user-defined unique gene sequences from complex eukaryotic genomes comprising:
isolating genomic from a human or animal;
shearing the genomic DNA into fragments, wherein the shearing is physical shearing selected from sonication, nebulization, or a combination thereof;
repairing the genomic DNA fragment, wherein repairing the genomic DNA fragments includes the addition of 3′-A extensions to the genomic DNA fragments;
ligating a plurality of adapters to the genomic DNA fragments, wherein the adaptors are blunt-end ligated to the genomic DNA fragments, and wherein the adapters have a 3′-T extension, do not substantially self ligate, are unique relative to the DNA genome, and are complimentary to one another, and wherein the adaptors have the nucleotide sequences according to SEQ ID NOs: 1 and 2;
hybridizing the genomic DNA fragments to oligonucleotides of interest of a high density long oligonucleotide microarray;
eluting of the genomic DNA fragments bound to oligonucleotides of interest on the microarray;
amplifying the eluted DNA fragments; and
resequencing of the eluted DNA fragments.
